(19) United States
(12) Patent Application Publication
(10) Pub. No.: US 2010/0152503 A1
(43) Pub. Date: Jun. 17, 2010

(54) Copper-Substituted Chromium Oxide Compositions, Their Preparation, and Their Use as Catalysts and Catalyst Precursors

(76) Inventors: Velliyur Nott Mallikarjuna Rao, Wilmington, DE (US); H. David Rosenfeld, Drumore, PA (US); Allen Sievert, Elkton, MD (US); Shekhar Subramoney, Hockessin, DE (US)

Correspondence Address: Heiser, David E.;E. I. Du Pont De Nemours And Company, Legal Patent Center, 4417 Lancaster Pike, Wilmington, DE, 19805, US

(21) Appl. No.: 11/988,256

(22) PCT Filed: Aug. 4, 2006
(86) PCT No.: PCT/US2006/030532

§ 371 Date: Apr. 16, 2009

Related U.S. Application Data
(60) Provisional application No. 60/706,159, filed on Aug. 5, 2005.

(51) Int. Cl. C07C 17/358; C07C 17/00; C07C 17/37; C01G 37/02; B01J 23/26

(52) U.S. Cl. 570/151; 570/123; 570/169; 570/163; 423/595; 502/318

(57) ABSTRACT

A crystalline alpha-chromium oxide where from about 0.05 atom % to about 5 atom % of the chromium atoms in the alpha-chromium oxide lattice are replaced by divalent copper (Cu⁺²) atoms is disclosed. Also disclosed is a chromium-containing catalyst composition comprising as a chromium-containing component the crystalline copper-substituted alpha-chromium oxide; and methods for preparing a composition comprising the crystalline copper-substituted alpha-chromium oxide. One method involves (a) co-precipitating a solid by adding ammonium hydroxide to an aqueous solution of a soluble copper salt and a soluble trivalent chromium salt that contains at least three moles of nitrate per mole of chromium in the solution and has a copper concentration of from about 0.05 atom % to about 5 atom % of the total concentration of copper and chromium in the solution; and after at least three moles of ammonium per mole of chromium in the solution has been added to the solution, (b) collecting the co-precipitated solid formed in (a); (c) drying the collected solid; and (d) calcining the dried solid. Another method involves (a) preparing an aqueous solution of a soluble copper salt and a soluble trivalent chromium salt that contains a copper concentration of from about 0.05 atom % to about 5 atom % of the total concentration of copper and chromium in the solution, (b) evaporating the solution to dryness, and (c) calcining the dried solid. Also disclosed is a chromium-containing catalyst composition comprising a chromium-containing component prepared by treating the crystalline copper-substituted alpha-chromium oxide with a fluorinating agent; and a process for changing the fluorine distribution (i.e., content and/or arrangement) in a hydrocarbon or halogenated hydrocarbon in the presence of a catalyst. The process involves using as the catalyst a composition comprising the crystalline copper-substituted alpha-chromium oxide and/or the treated copper-substituted alpha-chromium oxide.

### FIELD OF THE INVENTION

This invention relates to chromium-containing compositions and their preparation and use for the catalytic processing of hydrocarbons and/or halogenated hydrocarbons.

### BACKGROUND

It is well known that α-Cr₂O₃ and α-Fe₂O₃ have in common the structure of α-Al₂O₃ (corundum) with the M⁺³ ions occupying octahedral sites in the hexagonally close-packed oxide lattice. In contrast, Cu₂O (Cuprite) has Cu coordinated with 2 oxygen atoms in a cubic structure comprised of two interpenetrating Cu—O networks similar to the Si—O networks in Cristobalite. CuO (tenorite) is a monoclinic crystal structure with Cu atoms located in distorted octahedra with 4 co-planar oxygen atoms at 1.947 Å, and 2 apical oxygen atoms at 2.766 Å. These basic structures are described in standard treatises; see, for example, pages 538, 543-545, and 550 of Structural Inorganic Chemistry by A. F. Wells, 5^{th} ed. Clarendon Press, Oxford, UK (1986). γ-Chromium oxide (CrO_{2.44}) is described in Wilhelmi, Acta Chemica Scandinavica, Vol. 22, pages 2565-2573 (1968).

Numerous mixed metal oxides have been prepared in which the cation sites of the lattice are occupied by different metal ions. For example, solid solutions of the type (CrₓFe₁₋ₓ)₂O₃ are known where 0<x<1. These materials have been prepared by standard ceramic or sol-gel techniques as described by Music, et al. in J. Materials Science, Vol. 31, pages 4067-4076 (1996) and by Bhattacharya, et al. in J. Materials Science, Vol. 32, pages 577-560 (1997).

Various mixed Cr—Cu oxides including copper chromite, copper chromate and copper dichromate are known.

Certain metal oxides are used as catalysts and/or catalyst precursors in the manufacture of fluorinated hydrocarbons. Chromium(III) oxide in particular is useful as it has been found that it may be fluorinated by HF at elevated temperature to a give mixture of chromium fluoride and chromium oxyfluoride species which are active catalysts for conversion of C—Cl bonds to C—F bonds in the presence of HF. This conversion of C—Cl bonds to C—F bonds by the action of HF, known generally as halogen exchange, is a key step in many fluorocarbon manufacturing processes.

Chromium oxide compositions useful as catalyst precursors may be prepared in various ways or may take various forms. Chromium oxide suitable for vapor phase fluorination reactions may be prepared by reduction of Cr(VI) trioxide, by dehydration of Guignet's green, or by precipitation of Cr(III) salts with bases (see U.S. Pat. No. 3,258,500). Another useful form of chromium oxide is hexagonal chromium oxide hydroxide with low alkali metal ion content as disclosed in U.S. Pat. No. 3,978,145. Compounds such as MF₄ (M=Ti, Th, Ce), MF₃ (M=Al, Fe, Y), and MF₂ (M=Ca, Mg, Sr, Ba, Zn) have been added to hexagonal chromium oxide hydroxide to increase catalyst life as disclosed in U.S. Pat. No. 3,992,325. A form of chromium oxide that is a precursor to a particularly active fluorination catalyst is that prepared by pyrolysis of ammonium dichromate as disclosed in U.S. Pat. No. 5,036,036.

The addition of other compounds (e.g., other metal salts) to supported and/or unsupported chromium-based fluorination catalysts has been disclosed. Australian Patent Document No. AU-A-80340/94 discloses bulk or supported catalysts based on chromium oxide (or oxides of chromium) and at least one other catalytically active metal (e.g., Mg, V, Mn, Fe, Co, Ni, or Zn), in which the major part of the oxide(s) is in the crystalline state (and when the catalyst is a bulk catalyst, its specific surface, after activation with HF, is at least 8 m²/g). The crystalline phases disclosed include Cr₂O₃, CrO₂, NiCrO₃, NiCrO₄, NiCr₂O₄, MgCrO₄, ZnCr₂O₄ and mixtures of these oxides. Australian Patent Document AU-A-29972/92 discloses a mass catalyst based on chromium and nickel oxides in which the Ni/Cr atomic ratio is between 0.05 and 5. U.S. Patent Application Publication No. US2001/0011061 A1 discloses chromia-based fluorination catalysts (optionally containing Mg, Zn, Co, and Ni) in which the chromia is at least partially crystalline. Fluorinated catalysts containing cobalt and chromium in combination (e.g. impregnated on a support) are among those disclosed in U.S. Pat. No. 5,185,482. U.S. Pat. No. 5,559,069 discloses homogeneously dispersed multiphase catalyst compositions characterized by dispersed phases of certain divalent metal fluorides (certain fluorides of Mn, Co, Zn, Mg, and/or Cd) and certain trivalent metal fluorides (fluorides of Al, Ga, V, and for Cr).

There remains a need for halogen exchange catalysts that can be used for processes such as the selective fluorination and chlorofluorination of saturated and unsaturated hydrocarbons, hydrochlorocarbons, hydrochlorofluorocarbons, and chlorofluorocarbons, the fluorination of unsaturated fluorocarbons, the isomerization and disproportionation of fluorinated organic compounds, the dehydrofluorination of hydrofluorocarbons, and the chlorodefluorination of fluorocarbons.

### SUMMARY OF THE INVENTION

This invention provides a crystalline alpha-chromium oxide where from about 0.05 atom % to about 5 atom % of the chromium atoms in the alpha-chromium oxide lattice are replaced by divalent copper (Cu⁺²) atoms, and a chromium-containing catalyst composition comprising as a chromium-containing component said crystalline copper-substituted alpha-chromium oxide.

This invention also provides a co-precipitation method for preparing a composition comprising said crystalline copper-substituted alpha-chromium oxide. The method comprises (a) co-precipitating a solid by adding ammonium hydroxide (aqueous ammonia) to an aqueous solution of a soluble copper salt and a soluble trivalent chromium salt that contains at least three moles of nitrate (i.e., NO₃⁻) per mole of chromium (i.e., Cr³⁺) in the solution and has a copper concentration of from about 0.05 atom % to about 5 atom % of the total concentration of copper and chromium in the solution; and after at least three moles of ammonium (i.e., NH₄⁺) per mole of chromium (i.e., Cr³⁺) in the solution has been added to the solution, (b) collecting the co-precipitated solid formed in (a); (c) drying the collected solid; and (d) calcining the dried solid.

This invention also provides a thermal method for preparing a composition comprising said crystalline copper-substituted alpha-chromium oxide. The method comprises (a) preparing an aqueous solution of a soluble copper salt and a soluble trivalent chromium salt that contains a copper concentration of from about 0.05 atom % to about 5 atom % of the total concentration of copper and chromium in the solution; (b) evaporating the solution to dryness; and (c) calcining the dried solid.

This invention also provides a chromium-containing catalyst composition comprising a chromium-containing component prepared by treating said crystalline copper-substituted alpha-chromium oxide with a fluorinating agent (e.g., hydrogen fluoride).

This invention also provides a process for changing the fluorine distribution (i.e., content and/or arrangement) in a hydrocarbon or halogenated hydrocarbon in the presence of a catalyst. The process is characterized by using as the catalyst a composition comprising at least one chromium-containing component selected from the group consisting of said crystalline copper-substituted alpha-chromium oxides and said treated copper-substituted alpha-chromium oxides.

### BRIEF DESCRIPTION OF THE DRAWING

FIG. 1 represents a plot of the radial distribution function (i.e., the probability of finding an atom at a certain distance, r, from a central atom) associated with the local atomic structure around (a) a copper central atom in Cu₂O, (b) a copper central atom in CuO, (c) a copper central atom in Cu₂Cr₂O₅, (d) a chromium central atom in Cr₂O₃, (e) copper in a sample of copper-substituted alpha-chromium oxide nominally containing 1 atom % copper and (f) copper in a sample of copper-substituted alpha-chromium oxide nominally containing 2 atom % copper.

### DETAILED DESCRIPTION

New compositions of this invention comprise copper-substituted alpha-chromium oxide containing from about 0.05 atom % to about 5 atom % copper based on the total of the copper and chromium in the alpha-chromium oxide which retains the corundum structure. This invention includes a catalytic composition comprising said crystalline copper-substituted α-Cr₂O₃. The crystalline copper-substituted alpha-chromium oxides have the general formula α-CuₓCr₂₋ₓO₃ where x=0.001-0.10. However, it is understood that inasmuch as the copper component of these crystalline oxides is generally divalent, the oxygen component may average slightly less than three atoms per formula unit in order to maintain charge neutrality (i.e., there is a small percentage of vacant oxygen sites). Of note are embodiments containing at least 1 atom % copper based on the total of the copper and chromium in the alpha-chromium oxide (e.g., from about 2 atom % to about 3 atom % copper based on the total of the copper and chromium in the alpha-chromium oxide).

The compositions of the present invention may be prepared by co-precipitation. In the typical co-precipitation technique, an aqueous solution of copper(II) salts and chromium(III) salts is prepared. The relative concentrations of copper and chromium salts in the aqueous solution is dictated by the bulk atom percent copper relative to chromium desired in the final catalyst. The concentration of chromium salt in the aqueous solution is typically in the range of from about 0.3 to about 3 molar (moles per liter) with about 0.75-1.5 molar being a preferred concentration. Chromium(III) salts suitable for preparation of the aqueous solution are the nitrate, sulfate, acetate, formate, oxalate, phosphate, bromide, and chloride and various hydrated forms of these salts. Other chromium(III) salts that are useful for the preparation of the aqueous solutions include hexacoordinate complexes of the formula [CrL_{6-z}A_{z}]^{+3-z} where each L is a neutral (i.e., uncharged) ligand selected from the group consisting of H₂O, NH₃, C₁-C₄ primary, secondary, or tertiary organic amines, a C₁-C₄ alkyl nitriles, or pyridine, where each A is an anionic ligand selected from the group consisting of fluoride, chloride, bromide, iodide, hydroxide, nitrite, and nitrate, and where z has a value of from 0 to 3 inclusive. Included are neutral bidentate ligands such as ethylene diamine which are equivalent to two L in that they may occupy two coordination sites. Also included are anionic bidentate ligands such as C₁-C₄ carboxylate which may occupy two coordination sites. Also included are dianionic ligands such as sulfate which are equivalent to two A ligands and may occupy more than one coordination site.

Chromium(VI) precursors, such as CrO₃, though not preferred, may be used but require reduction to Cr(III) with a compound such as ethanol before precipitation.

Chromium(III) nitrate, or its hydrated forms such as [Cr(NO₃)₃(H₂O)₉], are the most preferred chromium(III) salt for preparation of said aqueous solution.

Copper(II) salts suitable for preparation of the aqueous solution are the nitrate, sulfate, formate, oxalate, bromide, and chloride and various hydrated forms of these salts. Copper(II) nitrate hydrate (e.g., [Cu(NO₃)₂(H₂O)_{2.5}]) is the most preferred copper(II) salt.

Of note are embodiments wherein the soluble copper and chromium salts are nitrates or hydrated nitrates.

The aqueous solution of the copper salts and chromium(III) salts is then treated with a base such as ammonium hydroxide (aqueous ammonia) to precipitate copper and chromium as the hydroxides. The addition of ammonium hydroxide to the aqueous solution of copper and chromium(III) salts is typically carried out gradually over a period of 1 to 12 hours. The pH of the solution is monitored during the addition of base. The final pH is typically in the range of 6.0 to 11.0, preferably from about 7.5 to about 9.0, and most preferably from about 8.0 to 8.7. The precipitation of the copper hydroxide/chromium hydroxide mixture is typically carried out at a temperature of about 15° C. to about 60° C., preferably from about 20° C. to about 40° C. After the ammonium hydroxide is added, the mixture is typically stirred for up to 24 hours.

Optionally, excess ammonium nitrate (i.e., more than three moles of ammonium nitrate per mole of chromium) may be added to the aqueous solution. For example, in addition to the ammonium nitrate already present from reaction of ammonium hydroxide with chromium nitrate, from about 0.1 mole to about 7.0 moles of additional ammonium nitrate per mole of chromium may be added to the solution before, during, or after the co-precipitation of the compositions.

After the ammonium nitrate is added to the mixture, it is preferably stirred for about 0.5 to ten hours (preferably for about one to five hours) at a temperature of from about 20° C. to about 60° C. The mixture is then dried and calcined as indicated below.

Other agents that serve this purpose include aqueous hydrogen peroxide (1% to 30% solutions), ozone, peroxy acids such as peroxyacetic acid, and ammonium persulfate. Agents such as halogens may be used but are not preferred. Agents containing alkali metals such as potassium persulfate or sodium perborate may also be used, but are not preferred.

After the precipitation of the mixture of copper and chromium hydroxides is complete, and the ammonium nitrate or other agents added if desired, the mixture is dried by evaporation.

After the copper and chromium hydroxide mixture has been dried, the residual nitrate salts are then decomposed by heating the solid from about 250° C. to about 350° C. The resulting solid is then calcined at temperature of from about 375° C. to about 1000° C., preferably from about 400° C. to about 900° C. The calcination is preferably carried out in the presence of oxygen, most preferably in the presence of air.

Compositions of this invention may also be prepared by a thermal method. In this method, a solution of the copper and chromium(III) salt is prepared as described for the co-precipitation technique. The mixed solution of the salts is then evaporated under atmospheric pressure or reduced pressure to give a solid. The solid is then placed in a furnace and the temperature raised gradually to decompose the salt. It is preferred to use the nitrate salts that decompose to the oxide. After decomposition of the nitrate salts is complete (about 350° C.), the increase in temperature is continued until the desired calcination temperature is reached. The desired calcination temperature is between about 450° C. to about 1000° C., a temperature of about 450° C. to about 900° C. being preferred. After the desired calcination temperature is reached, the solid is maintained at this temperature for an additional 8 to 24 hours, about 8 to about 12 hours being preferred. The decomposition and calcination is preferably carried out in the presence of oxygen, most preferably in the presence of air.

The metal oxide compositions of this invention may be characterized by well-established analytical techniques including X-Ray absorption spectroscopy (XAS), X-ray powder diffraction (XRD), transmission electron microscopy (TEM), and energy dispersive spectroscopy (EDS). EDS is an analytical tool available in conjunction with scanning or analytical TEM.

After calcination, the resulting copper-substituted crystallites are not visually distinguishable from α-Cr₂O₃ by TEM. Furthermore, X-ray and electron diffraction studies are entirely consistent with the α-Cr₂O₃ structure with some change in the lattice constants due to Cu(II) substituting for Cr(III) in the structure. The compositions are therefore concluded to have the general formula CuₓCr₂₋ₓO₃ where x=0.001-0.10. The EDS analysis from a sample containing 2 atom % Cu shows a uniform presence of Cu throughout the chromia particles, whereas this signal is absent in the chromia particles of a control sample when it is similarly analyzed.

XAS and XRD data were obtained for compositions that were nominally 100% Cr (no copper added), Cr99%/Cu1%, and Cr98%/Cu2%. XAS and XRD analysis clearly show that copper is substituted into α-Cr₂O₃. XRD results for Cr98%/Cu2% are shown in Table A (the numbers in the parentheses represent the standard deviations associated with the respective determinations). Diffraction peaks having d-spacings of 3.1335, 1.9188, and 1.6373 are due to a silicon internal standard added to the sample for calibration of the diffractometer. The peak at 1.7814 is due to the diffractometer sample holder. All other diffraction peaks can be indexed to the α-Cr₂O₃ structure with small adjustments to the lattice constants.

TABLE A
XRD Results for a Cu-Substituted alpha-Cr₂O₃ Composition
that is Nominally 98 atom % Cr/2 atom % Cu

| D (Angstroms) | Height | FWHM a. |
|---|---|---|
| 3.6300(0.0027) | 179(3) | 0.482(0.009) |
| 3.1335(0.0011) | 73(4) | 0.115(0.010) |
| 2.6656(0.0010) | 354(4) | 0.440(0.006) |
| 2.4780(0.0004) | 610(9) | 0.237(0.003) |
| 2.2608(0.0018) | 40(2) | 0.247(0.021) |
| 2.1731(0.0005) | 226(6) | 0.234(0.006) |
| 2.0571(0.0006) | 123(5) | 0.207(0.011) |
| 1.9188(0.0004) | 51(4) | 0.108(0.012) |
| 1.8159(0.0011) | 111(2) | 0.553(0.014) |
| 1.7814(0.0011) | 26(3) | 0.232(0.038) |
| 1.6714(0.0002) | 526(8) | 0.304(0.004) |
| 1.6373(0.0003) | 69(4) | 0.107(0.010) |
| 1.5793(0.0021) | 22(1) | 0.607(0.050) |

a. FWHM means full width at half maximum.

If Cu(II) substitutes for Cr(III) in the α-Cr₂O₃ phase, it is expected to be in a distorted octahedral coordination environment. We do not expect Cu2+ to be found in a regular octahedral environment with 6 equal length Cu—O bonds, because of the Jahn-Teller distortion of the valence orbitals. XAS results from the Cr—K edge of the samples indicate that all Cr is present as Cr³⁺ and is octahedrally coordinated.

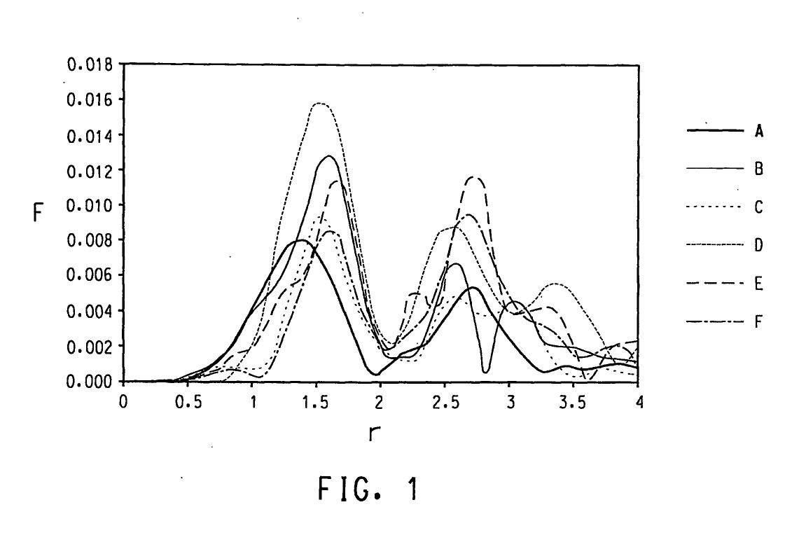

FIG. 1 shows the radial distribution function (RDF) for five materials. The radial distribution function represents the probability of finding an atom at a certain distance, r, from a central atom. These probabilities are weighted by factors that depend on the type of atom. Thus an RDF is a representation of local atomic structure around the central atom. An RDF is obtained by Fourier transform of the extended x-ray absorption fine structure (EXAFS) data, and may be represented by a plot of the dimensionless Fourier transform magnitude, F, versus the pair separation distance in angstroms. In simplified terms, one might view a peak in an RDF plot as indicative of a distance at which there is a coordination sphere around the central atom. A small difference is expected between the actual separation distance and the “r” shown in a plot when no correction is made to account for the phase shift on backscattering of excited electrons. In FIG. 1, F is plotted against the pair separation distance, r (shown in angstroms, uncorrected for phase shift) for each of the five materials. Included in FIG. 1 are curve A representing the local structure around copper in Cu₂O, curve B representing the local structure around copper in CuO, curve C representing the local structure around copper in Cu₂Cr₂O₅, curve D representing the local structure around chromium in α-Cr₂O₃. Also included in FIG. 1 is curve E representing the local structure around copper in the copper-substituted alpha-chromium oxide with a nominal composition of 99% chromium and 1% copper, and curve F representing the local structure around copper in the copper-substituted alpha-chromium oxide with a nominal composition of 98% chromium and 2% copper. XAS near edge spectroscopy indicates Cu is present as Cu²⁺ in the copper-substituted alpha-chromium oxides, so cuprous chromium oxides need not be considered. The curves (E & F) in FIG. 1 representing the local structure around copper in the copper-substituted alpha-chromium oxides, indicate that the local atomic structure around Cu in these samples bears no resemblance to that of expected common copper oxide phases, or known mixed Cr—Cu oxides, rather it is very similar to that of Cr in the α-Cr₂O₃ phase with distortions due to the distorted Cu²⁺ valence electron structure. These distortions manifest themselves in the observed lattice constants for the copper-substituted-chromia phase.

The calcined chromium oxide compositions of the present invention may be formed into various shapes such as pellets, granules, and extrudates for use in packing reactors. It may also be used in powder form.

The compositions of this invention may further comprise one or more additives in the form of metal compounds that alter the selectivity or activity of the crystalline copper-substituted alpha-chromium oxides or the fluorinated metal oxide catalysts containing copper and chromium. Suitable additives may be selected from the group consisting of fluorides, oxides, or oxyfluoride compounds of Mg, Ca, Sc, Y, La, Ti, Zr, Hf, V, Nb, Ta, Mo, W, Mn, Re, Fe, Ru, Co, Rh, Ir, Ni, Pd, Pt, Ag, Au, Ce, and Zn.

The total content of the additive(s) in the compositions of the present invention may be from about 0.05 atom % to about 15 atom % based on the total metal content of the compositions. The additives may be incorporated into the compositions of the present invention by standard procedures such as by impregnation.

Typically, the calcined compositions will be pre-treated with a fluorinating agent prior to use as catalysts for changing the fluorine distribution of hydrocarbons and/or halogenated hydrocarbon compounds. Typically this fluorinating agent is HF though other materials may be used such sulfur tetrafluoride, carbonyl fluoride, and fluorinated hydrocarbon compounds such as trichlorofluoromethane, dichlorodifluoromethane, chlorodifluoromethane, trifluoromethane, or 1,1,2-trichlorotrifluoroethane. This pretreatment can be accomplished, for example, by placing the catalyst in a suitable container which can be the reactor to be used to perform the process in the instant invention, and thereafter, passing HF over the dried, calcined catalyst so as to partially saturate the catalyst with HF. This is conveniently carried out by passing HF over the catalyst for a period of time, for example, about 0.1 to about 10 hours at a temperature of, for example, about 200° C. to about 450° C. Nevertheless, this pre-treatment is not essential.

As noted above catalysts provided in accordance with this invention may be used for changing the fluorine distribution in hydrocarbons and/or halogenated hydrocarbons. The fluorine distribution in a hydrocarbon or a halogenated hydrocarbon may be changed by increasing the fluorine content of the hydrocarbon or the halogenated hydrocarbon. The fluorine distribution of a halogenated hydrocarbon may also be changed by decreasing the fluorine content of the halogenated hydrocarbon and/or rearranging the placement of fluorine atoms on the carbon atoms of the halogenated hydrocarbon. Of note are processes where the fluorine distribution in halogenated hydrocarbons containing from one to twelve carbon atoms is changed, particularly processes where the fluorine distribution in halogenated hydrocarbons containing from one to six carbon atoms is changed. Also of note are processes where the fluorine content of hydrocarbons containing from one to twelve carbon atoms is increased, particularly processes where the fluorine content in hydrocarbons containing one to six carbon atoms is increased. Processes for changing the fluorine distribution in halogenated hydrocarbons include fluorination, chlorofluorination, isomerization, disproportionation, dehydrofluorination and chlorodefluorination. The processes of this invention are characterized by using as the catalyst a composition comprising at least one chromium-containing component selected from the group consisting of the crystalline copper-substituted alpha-chromium oxide described above and said copper-substituted alpha-chromium oxide which has been treated with a fluorinating agent.

Typical of saturated halogenated hydrocarbons suitable for fluorination, chlorofluorination, isomerization, disproportionation, dehydrofluorination and chlorodefluorination processes are those which have the formula CₙHₐBr_{b}Cl_{c}F_{d}, wherein n is an integer from 1 to 6, a is an integer from 0 to 12, b is an integer from 0 to 4, c is an integer from 0 to 13, d is an integer from 0 to 13, the sum of b, c and d is at least 1 and the sum of a, b, c, and d is equal to 2n+2, provided that n is at least 2 for isomerization, disproportionation and dehydrofluorination processes, a is at least one for dehydrofluorination processes, b is 0 for chlorodefluorination processes, b+c is at least 1 for fluorination processes and is 0 for dehydrofluorination processes, a+b+c is at least 1 for fluorination, chlorofluorination, isomerization, disproportionation and dehydrofluorination processes and d is at least 1 for isomerization, disproportionation, dehydrofluorination and chlorodefluorination processes. Typical of unsaturated halogenated hydrocarbons suitable for fluorination, chlorofluorination, isomerization, disproportionation, and chlorodefluorination processes are those which have the formula CₚHₑBr_{f}Cl_{g}Fₕ, wherein p is an integer from 2 to 6, e is an integer from 0 to 10, f is an integer from 0 to 2, g is an integer from 0 to 12, h is an integer from 0 to 11, the sum of f, g and h is at least 1 and the sum of e, f, g, and h is equal to 2p, provided that f is 0 for chlorodefluorination processes, e+f+g is at least 1 for isomerization and disproportionation processes and h is at least 1 for isomerization, disproportionation and chlorodefluorination processes. Typical of saturated hydrocarbons suitable for chlorofluorination are those which have the formula C_{q}Hᵣ where q is an integer from 1 to 6 and r is 2q+2. Typical of unsaturated hydrocarbons suitable for fluorination and chlorofluorination are those which have the formula where i is an integer from 2 to 6 and j is 21.

### Fluorination

Included in this invention is a process for increasing the fluorine content of a halogenated hydrocarbon compound or an unsaturated hydrocarbon compound by reacting said compound with hydrogen fluoride in the vapor phase in the presence of a catalyst composition comprising at least one chromium-containing component selected from the group consisting of the crystalline copper-substituted alpha-chromium oxide described above and said copper-substituted alpha-chromium oxide which has been treated with a fluorinating agent. The catalyst composition may optionally contain additional components such as additives to alter the activity and selectivity of the catalyst.

Halogenated hydrocarbon compounds suitable as starting materials for the fluorination process of this invention may be saturated or unsaturated. Saturated halogenated hydrocarbon compounds suitable for the fluorination processes of this invention include those of the general formula CₕHₐBr_{b}Cl_{c}F_{d}, wherein n is an integer from 1 to 6, a is an integer from 0 to 12, b is an integer from 0 to 4, c is an integer from 0 to 13, d is an integer from 0 to 13, and the sum of a, b, c, and d is equal to 2n+2, provided that b+c is at least 1. Unsaturated halogenated hydrocarbon compounds suitable for the fluorination processes of this invention include those of the general formula CₚHₑBr_{f}Cl_{g}Fₕ, wherein p is an integer from 2 to 6, e is an integer from 0 to 10, f is an integer from 0 to 2, g is an integer from 0 to 12, h is an integer from 0 to 11, the sum of f, g and h is at least 1 and the sum of e, f, g, and h is equal to 2p. Unsaturated hydrocarbons suitable for fluorination are those which have the formula CiHj where i is an integer from 2 to 6 and j is 21. The fluorine content of saturated compounds of the formula CₙHₐBr_{b}Cl_{c}F_{d}, unsaturated compounds of the formula CₚHₑBr_{f}Cl_{g}Fₕ and/or unsaturated compounds of the formula CᵢHⱼ may be increased by reacting said compounds with HF in the vapor phase in the presence of a catalyst composition comprising at least one chromium-containing component selected from the group consisting of the crystalline copper-substituted alpha-chromium oxide described above and said copper-substituted alpha-chromium oxide which has been treated with a fluorinating agent. Such a process is referred to herein as a vapor phase fluorination reaction.

The vapor phase fluorination reactions are typically conducted at temperatures of from about 150° C. to 500° C. For saturated compounds the fluorination is preferably carried out from about 175° C. to 400° C. and more preferably from about 200° C. to about 350° C. For unsaturated compounds the fluorination is preferably carried out from about 150° C. to 350° C. and more preferably from about 175° C. to about 300° C.

The vapor phase fluorination reactions are typically conducted at atmospheric and superatmospheric pressures. For reasons of convenience in downstream separations processes (e.g., distillation), pressures of up to about 30 atmospheres may be employed.

The vapor phase fluorination reactions are typically conducted in a tubular reactor. The reactor and its associated feed lines, effluent lines, and associated units should be constructed of materials resistant to hydrogen fluoride and hydrogen chloride. Typical materials of construction, well-known to the fluorination art, include stainless steels, in particular of the austenitic type, the well-known high nickel alloys, such as Monel® nickel-copper alloys, Hastelloy® nickel-based alloys and, Inconel® nickel-chromium alloys, and copper-clad steel.

The contact time in the reactor is typically from about 1 to about 120 seconds. Of note are contact times of from about 5 to about 60 seconds.

The amount of HF reacted with the unsaturated hydrocarbons or halogenated hydrocarbon compounds should be at least a stoichiometric amount. The stoichiometric amount is based on the number of Br and/or Cl substituents to be replaced by F in addition to one mole of HF to saturate the carbon-carbon double bond if present. Typically, the molar ratio of HF to the said compounds of the formulas CₙHₐBr_{b}Cl_{c}F_{d}, CₚHₑBr_{f}Cl_{g}Fₕ, CᵢHⱼ and can range from about 0.5:1 to about 100:1, preferably from about 2:1 to about 50:1, and more preferably from about 3:1 to about 20:1. In general, with a given catalyst composition, the higher the temperature and the longer the contact time, the greater is the conversion to fluorinated products. The above variables can be balanced, one against the other, so that the formation of higher fluorine substituted products is maximized.

Examples of saturated compounds of the formula CₙHₐBr_{b}Cl_{c}F_{d} which may be reacted with HF in the presence of the catalyst of this invention include CH₂Cl₂, CH₂Br₂, CHCl₃, CCl₄, C₂Cl₆, C₂BrCl₅, C₂Cl₅F, C₂Cl₄F₂, C₂Cl₃F₃, C₂Cl₂F₄, C₂ClF₅, C₂HCl₅, C₂HCl₄F, C₂HCl₃F₂, C₂HCl₂F₃, C₂HClF₄, C₂HBrF₄, C₂H₂Cl₄, C₂H₂Cl₃F, C₂H₂Cl₂F₂, C₂H₂ClF₃, C₂H₃Cl₃, C₂H₃Cl₂F, C₂H₃ClF₂, C₂H₄Cl₂, C₂H₄ClF, C₃Cl₆F₂, C₃Cl₅F₃, C₃Cl₄F₄, C₃Cl₃F₅, C₃HCl₇, C₃HCl₆F, C₃HCl₅F₂, C₃HCl₄F₃, C₃HCl₃F₄, C₃HCl₂F₅, C₃H₂Cl₆, C₃H₂BrCl₅, C₃H₂Cl₅F, C₃H₂Cl₄F₂, C₃H₂Cl₃F₃, C₃H₂Cl₂F₄, C₃H₂ClF₅, C₃H₃Cl₅, C₃H₃Cl₄F, C₃H₃Cl₃F₂, C₃H₃Cl₂F₃, C₃H₃ClF₄, C₃H₄Cl₄, C₄Cl₄Cl₄, C₄Cl₄Cl₆, C₄H₆Cl₆, C₄H₅Cl₄F₁ and C₆H₄Cl₈.

Specific examples of fluorination reactions of saturated halogenated hydrocarbon compounds which may be carried out under the conditions described above using the catalysts of this invention include the conversion of CH₂Cl₂ to CH₂F₂, the conversion of CHCl₃ to a mixture of CHCl₂F, CHClF₂, and CHF₃, the conversion of CH₃CHCl₂ to a mixture of CH₃CHClF and CH₃CHF₂, the conversion of CH₂ClCH₂Cl to a mixture of CH₃CHClF and CH₃CHF₂, the conversion of CH₃CCl₃ to a mixture of CH₃CCl₂F, CH₃CClF₂, and CH₃CF₃, the conversion of CH₂ClCF₃ to CH₂FCF₃, the conversion of CHCl₂CF₃ to a mixture of CHClFCF₃ and CHF₂CF₃, the conversion of CHClFCF₃ to CHF₂CF₃, the conversion of CHBrFCF₃ to CHF₂CF₃, the conversion of CCl₃CF₂CCl₃ to a mixture of CCl₂FCF₂CClF₂ and CClF₂CF₂CClF₂, the conversion of CCl₃CH₂CCl₃ to CF₃CH₂CClF₂ and CF₃CH₂CF₃, the conversion of CCl₃CH₂CHCl₂ to a mixture of CF₃CH₂CHF₂, CF₃CH═CHCl, and CF₃CH═CHF, the conversion of CF₃CCl₂CClF₂ to a mixture of CF₃CCl₂CF₃, and CF₃CClFCF₃, the conversion of CF₃CCl₂CF₃ to CF₃ClFCF₃, and the conversion of a mixture comprising CF₃CF₂CHCl₂ and CClF₂CF₂CHClF to a mixture of CF₃CF₂CHClF and CF₃CF₂CHF₂.

Examples of unsaturated compounds of the formula CₚHₑBr_{f}Cl_{g}Fₕ and CᵢHᵢ which may be reacted with HF in the presence of the catalysts of this invention include C₂Cl₄, C₂BrCl₃, C₂Cl₃F, C₂Cl₂F₂, C₂ClF₃, C₂F₄, C₂HCl₃, C₂HBrCl₂, C₂HCl₂F, C₂HClF₂, C₂HF₃, C₂H₂Cl₂, C₂H₂ClF, C₂H₂F₂, C₂H₃Cl, C₂H₃F, C₂H₄, C₃H₆, C₃H₅Cl, C₃H₄Cl₂, C₃H₃Cl₃, C₃H₂Cl₄, C₃HCl₅, C₃Cl₆, C₃Cl₅F, C₃Cl₄F₂, C₃Cl₃F₃, C₃Cl₂F₄, C₃ClF₅, C₃HF₅, C₃H₂F₄, C₃F₆, C₄Cl₈, C₄Cl₂F₆, C₄ClF₇, C₄H₂F₆, and C₄HClF₆.

Specific examples of fluorination reactions of unsaturated halogenated hydrocarbon compounds which may be carried out using the catalysts of this invention include the conversion of CHCl═CCl₂ to a mixture of CH₂ClCF₃ and CH₂FCF₃, the conversion of CCl₂═CCl₂ to a mixture of CHCl₂CF₃, CHClFCF₃, and CHF₂CF₃, the conversion of CCl₂═CH₂ to a mixture of CH₃CCl₂F, CH₃CClF₂, and CH₃CF₃, the conversion of CH₂═CHCl to a mixture of CH₃CHClF and CH₃CHF₂, the conversion of CF₂═CH₂ to CH₃CF₃, the conversion of CCl₂═CClCF₃ to a mixture of CF₃CHClCClF₂, CF₃CHClCF₃, and/or CF₃CCl═CF₂, the conversion of CF₃CF═CF₂ to CF₃CHFCF₃, the conversion of CF₃CH═CF₂ to CF₃CH₂CF₃, and the conversion of CF₃CH═CHF to CF₃CH₂CHF₂.

Of note is a catalytic process for producing a mixture of 2-chloro-1,1,1,3,3,3-hexafluoropropane (i.e., CF₃CHClCF₃ or HCFC-226da) and 2-chloro-pentafluoropropene (i.e., CF₃CCl═CF₂ or CFC-1215xc) by the fluorination of a hexahalopropene of the formula C₃Cl₆₋ₓFₓ, wherein x equals 0 to 4. Preferred hexahalopropenes of the formula C₃Cl₆₋ₓFₓ include 1,1,2-trichloro-3,3,3-trifluoro-1-propene (i.e., CCl₂═CClCF₃ or CFC-1213xa) and hexachloropropene (i.e., CCl₂═CClCCl₃). The mixture of HCFC-226da and CFC-1215xc is produced by reacting the above unsaturated compounds with HF in the vapor phase in the presence of the catalysts of this invention at temperatures from about 150° C. to about 400° C., preferably about 200° C. to about 350° C.

The amount of HF fed to the reactor should be at least a stoichiometric amount based on the number of Cl substituents in the C₃Cl₆₋ₓFₓ starting material(s). In the case of fluorination of CFC-1213xa, the stoichiometric ratio of HF to CFC-1213xa is 3:1. Preferred ratios of HF to C₃Cl₆₋ₓFₓ starting material(s) are typically in the range of about the stoichiometric ratio to about 25:1. Preferred contact times are typically in the range of from 1 to 60 seconds.

Further information on the fluorination of CFC-1213xa is provided in U.S. Patent Application 60/706,164 filed Aug. 5, 2005, and hereby incorporated by reference herein in its entirety.

Mixtures of saturated halogenated hydrocarbon compounds or mixtures of unsaturated hydrocarbons and/or halogenated hydrocarbon compounds may also be used in the vapor phase fluorination reactions as well as mixtures comprising both unsaturated hydrocarbons and halogenated hydrocarbon compounds. Specific examples of mixtures of saturated halogenated hydrocarbon compounds and mixtures of unsaturated hydrocarbons and unsaturated halogenated hydrocarbon compounds that may be subjected to vapor phase fluorination using the catalysts of this invention include a mixture of CH₂Cl₂ and CCl₂═CCl₂, a mixture of CCl₂FCClF₂ and CCl₃CF₃, a mixture of CCl₂═CCl₂ and CCl₂═CClCCl₃, a mixture of CH₂═CHCH₃ and CH₂═CClCH₃, a mixture of CH₂Cl₂ and CH₃CCl₃, a mixture of CHF₂CClF₂ and CHClFCF₃, a mixture of CHCl₂CCl₂CH₂Cl and CCl₃CHClCH₂Cl, a mixture of CHCl₂CH₂CCl₃ and CCl₃CHClCH₂Cl, a mixture of CHCl₂CHClCCl₃, CCl₃CH₂CCl₃, and CCl₃CCl₂CH₂Cl, a mixture of CHCl₂CH₂CCl₃ and CCl₃CH₂CCl₃, a mixture of and CF₃CH₂CCl₂F and CF₃CH═CCl₂, and a mixture of CF₃CH═CHCl and CF₃CH═CCl₂.

### Chlorofluorination

Included in this invention is a process for increasing the fluorine content of a halogenated hydrocarbon compound or a hydrocarbon compound by reacting said compound with hydrogen fluoride (HF) and chlorine (Cl₂) in the vapor phase in the presence of a catalyst composition comprising at least one chromium-containing component selected from the group consisting of the crystalline copper-substituted alpha-chromium oxide described above and said copper-substituted alpha-chromium oxide which has been treated with a fluorinating agent. The catalyst composition may optionally contain additional components such as another catalytically effective metal.

Halogenated hydrocarbon compounds suitable as starting materials for the chlorofluorination process of this invention may be saturated or unsaturated. Saturated halogenated hydrocarbon compounds suitable for the chlorofluorination processes of this invention include those of the general formula CₙHₐBr_{b}Cl_{c}F_{d}, wherein n is an integer from 1 to 6, a is an integer from 0 to 12, b is an integer from 0 to 4, c is an integer from 0 to 13, d is an integer from 0 to 13, the sum of b, c and d is at least 1 and the sum of a, b, c, and d is equal to 2n+2, provided that a+b+c is at least 1. Preferred chlorofluorination processes include those involving said saturated starting materials where a is at least 1. Saturated hydrocarbon compounds suitable for chlorofluorination are those which have the formula C_{q}Hᵣ where q is an integer from 1 to 6 and r is 2q+2. Unsaturated halogenated hydrocarbon compounds suitable for the chlorofluorination processes of this invention include those of the general formula CₚHₑBr_{f}Cl_{g}Fₕ, wherein p is an integer from 2 to 6, e is an integer from 0 to 10, f is an integer from 0 to 2, g is an integer from 0 to 12, h is an integer from 0 to 11, the sum of f, g and h is at least 1 and the sum of e, f, g, and h is equal to 2p. Unsaturated hydrocarbon compounds suitable for fluorination are those which have the formula CᵢHⱼ where i is an integer from 2 to 6 and j is 21. The fluorine content of saturated compounds of the formula CₙHₐBr_{b}Cl_{c}F_{d} and C_{q}Hᵣ and/or unsaturated compounds of the formula CₚHₑBr_{f}Cl_{g}Fₕ and CᵢHⱼ may be increased by reacting said compounds with HF and Cl₂ in the vapor phase in the presence of a catalyst composition comprising at least one chromium-containing component selected from the group consisting of the crystalline copper-substituted alpha-chromium oxide described above and said copper-substituted alpha-chromium oxide which has been treated with a fluorinating agent. Such a process is referred to herein as a vapor phase chlorofluorination reaction.

The conditions of the vapor phase chlorofluorination reactions are similar to those described above for vapor phase fluorination reactions in terms of the temperature ranges, contact times, pressures, and mole ratios of HF to the halogenated hydrocarbon compounds. The amount of chlorine (Cl₂) fed to the reactor is based on whether the halogenated hydrocarbon compounds fed to the reactor is unsaturated and the number of hydrogens in CₙHₐBr_{b}Cl_{c}F_{d}, C_{q}Hᵣ, CₚHₑBr_{f}Cl_{g}Fₕ, and that are to be replaced by chlorine and fluorine. One mole of Cl₂ is required to saturate a carbon-carbon double bond and a mole of Cl₂ is required for every hydrogen to be replaced by chlorine or fluorine. A slight excess of chlorine over the stoichiometric amount may be necessary for practical reasons, but large excesses of chlorine will result in complete chlorofluorination of the products. The ratio of Cl₂ to halogenated carbon compound is typically from about 1:1 to about 10:1.

Specific examples of vapor phase chlorofluorination reactions of saturated halogenated hydrocarbon compounds of the general formula CₙHₐBr_{b}Cl_{c}F_{d} and saturated hydrocarbon compounds of the general formula C_{q}Hᵣ which may be carried out using the catalysts of this invention include the conversion of C₂H₆ to a mixture containing CH₂ClCF₃, the conversion of CH₂ClCF₃ to a mixture of CHClFCF₃ and CHF₂CF₃, the conversion of CCl₃CH₂CH₂Cl to a mixture of CF₃CCl₂CClF₂, CF₃CCl₂CF₃, CF₃CClFCClF₂, and CF₃CClFCF₃, the conversion of CCl₃CH₂CHCl₂ to a mixture of CF₃CCl₂CClF₂, CF₃CCl₂CF₃, CF₃CClFCClF₂, and CF₃CClFCF₃, the conversion of CCl₃CHClCH₂Cl to a mixture of CF₃CCl₂CClF₂, CF₃CCl₂CF₃, CF₃CClFCClF₂, and CF₃CClFCF₃, the conversion of CHCl₂CCl₂CH₂Cl to a mixture of CF₃CCl₂CClF₂, CF₃CCl₂CF₃, CF₃CClFCClF₂, and CF₃CClFCF₃, the conversion of CCl₃CH₂CH₂Cl to a mixture of CF₃CCl₂CHF₂, CF₃CClFCHF₂, CF₃CClFCClF₂, and CF₃CCl₂CF₃, and the conversion of CCl₃CH₂CHCl₂ to a mixture of CF₃CCl₂CHF₂, CF₃CClFCHF₂, CF₃CClFCClF₂, and CF₃CCl₂CF₃.

Specific examples of vapor phase chlorofluorination reactions of unsaturated halogenated hydrocarbon compounds of the general formula CₚHₑBr_{f}Cl_{g}Fₕ and unsaturated hydrocarbon compounds of the general formula CᵢHⱼ which may be carried out using the catalysts of this invention include the conversion of C₂H₄ to a mixture of CCl₃CClF₂, CCl₂FCCl₂F, CClF₂CCl₂F, CCl₃CF₃, CF₃CCl₂F, and CClF₂CClF₂, the conversion of C₂Cl₄ to a mixture of CCl₃CClF₂, CCl₂FCCl₂F, CClF₂CCl₂F, CCl₃CF₃, CF₃CCl₂F, and CClF₂CClF₂, and the conversion of C₃H₆ or CF₃CCl═CCl₂ to a mixture of CF₃CCl₂CClF₂, CF₃CCl₂CF₃, CF₃CClFCClF₂, and CF₃CClFCF₃.

Of note is a catalytic process for producing a mixture of 1,2,2-trichloro-1,1,3,3,3-pentafluoropropane (i.e., CClF₂CCl₂CF₃ or CFC-215aa), 1,1,2-trichloro-1,2,3,3,3-pentafluoropropane (i.e., CCl₂FCClFCF₃ or CFC-215bb), 2,2-dichloro-1,1,1,3,3,3-hexafluoropropane (i.e., CF₃CCl₂CF₃ or CFC-216aa), 1,2-dichloro-1,1,1,3,3,3-hexafluoropropane (i.e., CClF₂CClFCF₃ or CFC-216ba), and 2-chloro-1,1,1,2,3,3,3-heptafluoropropane (i.e., CF₃CClFCF₃ or CFC-217ba), by the chlorofluorination of a hexahalopropene of the formula C₃Cl₆₋ₓFₓ, wherein x equals 0 to 4. Preferred hexahalopropenes of the formula C₃Cl₆₋ₓFₓ include 1,1,2-trichloro-3,3,3-trifluoro-1-propene (i.e., CCl₂═CClCF₃ or CFC-1213xa) and hexachloropropene (i.e., CCl₂═CClCCl₃). The mixture of CFC-215aa, -215bb, -216aa, -216ba, and -217ba is produced by reacting the above unsaturated compounds with Cl₂ and HF in the vapor phase in the presence of the catalysts of this invention at temperatures from about 150° C. to about 450° C., preferably about 250° C. to 400° C.

The amount of HF fed to the reactor should be at least a stoichiometric amount based on the number of Cl substitutents in the C₃Cl₆₋ₓFₓ starting material(s) and the desired composition of the final product. In the case of chlorofluorination of CFC-1213xa to a mixture of chlorofluoropropanes having an average number of fluorine substituents of six, the stoichiometric ratio of HF to CFC-1213xa is 3:1. Preferred ratios of HF to C₃Cl₆₋ₓFₓ starting material(s) are typically in the range of about the stoichiometric ratio to about 30:1, more preferably from about 8:1 to 25:1.

The amount of chlorine fed to the reactor should be at least a stoichiometric amount. Preferred molar ratios of Cl₂ to CFC-1213xa are from about 1:1 to about 5:1.

Of note are contact times of from about 5 seconds to about 60 seconds.

Further information on the chlorofluorination of CFC-1213xa is provided in U.S. Patent Applications 60/706,161 and 60/706,162 filed Aug. 5, 2005, and hereby incorporated by reference herein in their entirety.

Mixtures of saturated hydrocarbon compounds and saturated halogenated hydrocarbon compounds and mixtures of unsaturated hydrocarbon compounds and unsaturated halogenated hydrocarbon compounds as well as mixtures comprising both saturated and unsaturated compounds may be chlorofluorinated using the catalysts of the present invention. Specific examples of mixtures of saturated and unsaturated hydrocarbons and halogenated hydrocarbons that may be used include a mixture of CCl₂═CCl₂ and CCl₂═CClCCl₃, a mixture of CHCl₂CCl₂CH₂Cl and CCl₃CHClCH₂Cl, a mixture of CHCl₂CH₂CCl₃ and CCl₃CHClCH₂Cl, a mixture of CHCl₂CHClCCl₃, CCl₃CH₂CCl₃, and CCl₃CCl₂CH₂Cl, a mixture of CHF₂CH₂CF₃ and CHCl═CHCF₃, and a mixture of CH₂═CH₂ and CH₂═CHCH₃.

### Isomerization and Disproportionation

Included in this invention is a process for changing the fluorine distribution in a halogenated hydrocarbon compound by isomerizing said halogenated hydrocarbon compound in the presence of a catalyst composition comprising at least one chromium-containing component selected from the group consisting of the crystalline copper-substituted-chromium oxide described above and said copper-substituted alpha-chromium oxide which has been treated with a fluorinating agent.

Also included in this invention is a process for changing the fluorine distribution in a halogenated hydrocarbon compound by disproportionating said halogenated hydrocarbon compound in the vapor phase in the presence of a catalyst composition comprising at least one chromium-containing component selected from the group consisting of the crystalline copper-substituted alpha-chromium oxide described above and said copper-substituted alpha-chromium oxide which has been treated with a fluorinating agent.

Halogenated hydrocarbon compounds suitable as starting materials for the isomerization and disproportionation processes of this invention may be saturated or unsaturated. Saturated halogenated hydrocarbon compounds suitable for the isomerization and disproportionation processes of this invention include those of the general formula CₙHₐBr_{b}Cl_{c}F_{d}, wherein n is an integer from 2 to 6, a is an integer from 0 to 12, b is an integer from 0 to 4, c is an integer from 0 to 13, d is an integer from 1 to 13, and the sum of a, b, c, and d is equal to 2n+2, provided that a+b+c is at least 1. Unsaturated halogenated hydrocarbon compounds suitable for the isomerization and disproportionation processes of this invention include those of the general formula CₚHₑBr_{f}Cl_{g}Fₕ, wherein p is an integer from 2 to 6, e is an integer from 0 to 10, f is an integer from 0 to 2, g is an integer from 0 to 12, h is an integer from 1 to 11, and the sum of e, f, g, and h is equal to 2p, provided that the sum of e+f+g is at least 1.

In one embodiment of the present invention, the fluorine distribution of a halogenated hydrocarbon compound is changed by rearranging the H, Br, Cl, and F substituents in the molecule (typically to a thermodynamically preferred arrangement) while maintaining the same number of the H, Br, Cl, and F substituents, respectively. This process is referred to herein as isomerization.

In another embodiment of the present invention, the fluorine distribution of a halogenated hydrocarbon compound is changed by exchanging at least one F substituent of the halogenated hydrocarbon starting material with at least one H, Br and/or Cl substituent of another molecule of the halogenated hydrocarbon starting material so as to result in the formation of one or more halogenated hydrocarbon compounds having a decreased fluorine content compared to the halogenated hydrocarbon starting material and one or more halogenated hydrocarbon compounds having an increased fluorine content compared to the halogenated hydrocarbon starting material. This process is referred to herein as disproportionation.

In another embodiment of the present invention, both isomerization and disproportionation reactions may occur simultaneously.

Whether carrying out isomerization, disproportionation or both isomerization and disproportionation, the fluorine distribution of saturated compounds of the formula CₙHₐBr_{b}Cl_{c}F_{d} and/or unsaturated compounds of the formula CₚHₑBr_{f}Cl_{g}Fₕ may be changed in the presence of a catalyst composition comprising at least one chromium-containing component selected from the group consisting of the crystalline copper-substituted alpha-chromium oxide described above and said copper-substituted alpha-chromium oxide which has been treated with a fluorinating agent.

The isomerization and disproportionation reactions are typically conducted at temperatures of from about 150° C. to 500° C., preferably from about 200° C. to about 400° C. The contact time in the reactor is typically from about 1 to about 120 seconds and preferably from about 5 to about 60 seconds. The isomerization and disproportionation reactions may be carried out in the presence of an inert gas such as helium, argon, or nitrogen though this is not preferred. The isomerization and disproportionation reactions may be carried out in the presence of HF and HCl, but this is not preferred.

Specific examples of vapor phase isomerization reactions which may be carried out using the catalysts of this invention include the conversion of CClF₂CCl₂F to CCl₃CF₃, the conversion of CClF₂CClF₂ to CF₃CCl₂F, the conversion of CHF₂CClF₂ to CF₃CHClF, the conversion of CHF₂CHF₂ to CF₃CH₂F, the conversion of CF₃CClFCClF₂ to CF₃CCl₂CF₃, and the conversion of CF₃CHFCHF₂ to CF₃CH₂CF₃.

Specific examples of vapor phase disproportionation reactions which may be carried out using the catalysts of this invention include the conversion of CClF₂CClF₂ to a mixture of CClF₂CCl₂F, CCl₃CF₃, and CF₃CClF₂, and the conversion of CHClFCF₃ to a mixture of CHCl₂CF₃, and CHF₂CF₃.

Of note is a process for the conversion of a mixture of 2-chloro-1,1,2,2-tetrafluoroethane (i.e., CHF₂CClF₂ or HCFC-124a) and 2-chloro-1,1,1,2-tetrafluoroethane (i.e., CF₃CHClF or HCFC-124) to a mixture comprising 2,2-dichloro-1,1,1-trifluoroethane (i.e., CHCl₂CF₃ or HCFC-123) and 1,1,1,2,2-pentafluoroethane (i.e., CF₃CHF₂ or HFC-125) in addition to unconverted starting materials. The mixture comprising HFC-125 and HCFC-123 may be obtained in the vapor phase by contacting a mixture of HCFC-124a and -124 over the catalysts of this invention optionally in the presence of a diluent selected from the group consisting of HF, HCl, nitrogen, helium, argon, and carbon dioxide. The disproportionation is preferably conducted at about 150° C. to about 400° C., more preferably about 250° C. to about 350° C. If used, the diluent gas, may be present in a molar ratio of diluent to haloethane of from about 1:1 to about 5:1. Of note are contact times of from about 10 seconds to about 60 seconds.

### Dehydrofluorination

Included in this invention is a process for decreasing the fluorine content of a halogenated hydrocarbon compound by dehydrofluorinating said halogenated hydrocarbon compound in the presence of a catalyst composition comprising at least one chromium-containing component selected from the group consisting of the crystalline copper-substituted alpha-chromium oxide described above and said copper-substituted alpha-chromium oxide which has been treated with a fluorinating agent.

Halogenated hydrocarbon compounds suitable as starting materials for the dehydrofluorination process of this invention are typically saturated. Saturated halogenated hydrocarbon compounds suitable for the dehydrofluorination processes of this invention include those of the general formula CₙHₐF_{d}, wherein n is an integer from 2 to 6, a is an integer from 1 to 12, d is an integer from 1 to 13, and the sum of a and d is equal to 2n+2. The fluorine content of saturated compounds of the formula CₙHₐF_{d} may be decreased in the presence of a catalyst composition comprising at least one chromium-containing component selected from the group consisting of the crystalline copper-substituted alpha-chromium oxide described above and said copper-substituted alpha-chromium oxide which has been treated with a fluorinating agent. This decrease in fluorine content is typically associated with removal of hydrogen fluoride (HF) from the molecule and is referred to herein as dehydrofluorination.

The dehydrofluorination reactions are typically conducted at temperatures of from about 200° C. to about 500° C., preferably from about 300° C. to about 450° C. The contact time in the reactor is typically from about 1 to about 360 seconds. Of note are contact times of from about 5 to about 120 seconds. Carrying out the dehydrofluorination reactions in the presence of an inert gas such as helium, argon, or nitrogen promotes the dissociation of the fluorinated carbon compound, but this practice can also lead to difficulties in separation and is not preferred.

The product of dehydrofluorination reaction consists of HF and the unsaturated fluorinated carbon compound resulting from loss of HF from the starting material. Specific examples of vapor phase dehydrofluorination reactions which may be carried out using the catalysts of this invention include the conversion of CH₃CHF₂ to CH₂═CHF, the conversion of CH₃CF₃ to CH₂═CF₂, the conversion of CF₃CH₂F to CF₂═CHF, the conversion of CHF₂CH₂CF₃ to CHF═CHCF₃, the conversion of CHF₂CHFCF₃ to CHF═CFCF₃, and the conversion of CF₃CH₂CF₃ to CF₃CH═CF₂.

Of note is a catalytic process for producing fluoroethene (i.e., CH₂═CHF or vinyl fluoride) by the dehydrofluorination of a 1,1-difluoroethane (i.e., CHF₂CH₃ or HFC-152a). A mixture comprising vinyl fluoride and unconverted HFC-152a may be obtained in the vapor phase by contacting HFC-152a over the catalysts of this invention optionally in the presence of a diluent selected from the group consisting of HF, nitrogen, helium, argon, and carbon dioxide. The dehydrofluorination is preferably conducted at about 150° C. to about 400° C., more preferably about 250° C. to about 350° C. If used, the diluent gas, may be present in a molar ratio of diluent to haloethane of from about 1:1 to about 5:1. Of note are contact times of from about 10 seconds to about 60 seconds.

### Chlorodefluorination

Included in this invention is a process for decreasing the fluorine content of a halogenated hydrocarbon compound by reacting said halogenated hydrocarbon compound with hydrogen chloride (HCl) in the vapor phase in the presence of a catalyst composition comprising at least one chromium-containing component selected from the group consisting of the crystalline copper-substituted alpha-chromium oxide described above and said copper-substituted alpha-chromium oxide which has been treated with a fluorinating agent.

Halogenated hydrocarbon compounds suitable as starting materials for the chlorodefluorination processes of this invention may be saturated or unsaturated. Saturated halogenated hydrocarbon compounds suitable for the chlorodefluorination processes of this invention include those of the general formula CₙHₐCl_{c}F_{d}, wherein n is an integer from 1 to 6, a is an integer from 0 to 12, c is an integer from 0 to 13, d is an integer from 1 to 13, and the sum of a, c and d is equal to 2n+2. Unsaturated halogenated hydrocarbon compounds suitable for the chlorodefluorination processes of this invention include those of the general formula CₚHₑCl_{g}Fₕ, wherein p is an integer from 2 to 6, e is an integer from 0 to 10, g is an integer from 0 to 12, h is an integer from 1 to 11, and the sum of e, g, and h is equal to 2p. The fluorine content of saturated compounds of the formula CₙHₐCl_{c}F_{d} and/or unsaturated compounds of the formula CₚHₑCl_{g}Fₕ may be decreased by reacting said compounds with HCl in the vapor phase in the presence of a catalyst composition comprising at least one chromium-containing component selected from the group consisting of the crystalline copper-substituted alpha-chromium oxide described above and said copper-substituted alpha-chromium oxide which has been treated with a fluorinating agent. Such a process is referred to herein as a vapor phase chlorodefluorination reaction. Chlorodefluorination is disclosed in U.S. Pat. No. 5,345,017 and U.S. Pat. No. 5,763,698 and the teachings of these two patents are hereby incorporated herein by reference.

The chlorodefluorination reactions are typically conducted at temperatures of from about 250° C. to 450° C., preferably from about 300° C. to about 400° C. The contact time in the reactor is typically from about 1 to about 120 seconds. Of note are contact times of from about 5 to about 60 seconds. The reactions are most conveniently carried out at atmospheric or superatmospheric pressure.

Chlorodefluorinations involving saturated halogenated hydrocarbons are of particular note. The molar ratio of HCl to the saturated halogenated hydrocarbon compound is typically from about 1:1 to about 100:1, preferably from about 3:1 to about 50:1, and most preferably from about 4:1 to about 30:1. In general, with a given catalyst composition, the higher the temperature, the longer the contact time, and the greater the molar ratio of HCl to saturated halogenated hydrocarbon compound, the greater is the conversion to compounds having lower fluorine content. The above variables can be balanced, one against the other, so that the formation of chlorine-substituted products is maximized.

The product of chlorodefluorination reactions typically comprise unreacted HCl, HF, unconverted starting material, and saturated halogenated hydrocarbon compounds having a lower fluorine content than the starting material by virtue of the substitution of one or more fluorine substituents for chlorine. Specific examples of vapor phase chlorodefluorination reactions which may be carried out using the catalysts of this invention include the conversion of CHF₃ to a mixture of CHCl₃, CHCl₂F, and CHClF₂, the conversion of CClF₂CClF₂ to a mixture of CCl₃CCl₃, CCl₃CCl₂F, CCl₃CClF₂, CCl₂FCCl₂F, CClF₂CCl₂F, and CCl₃CF₃, the conversion of CF₃CClF₂ to a mixture of CCl₃CCl₃, CCl₃CCl₂F, CCl₃CClF₂, CCl₂FCCl₂F, CClF₂CCl₂F, CCl₃CF₃, CClF₂CClF₂, and CF₃CCl₂F, the conversion of CF₃CCl₂CF₃ to a mixture of CF₃CCl₂CClF₂, CF₃CCl₂CCl₂F, CF₃CCl₂CCl₃, and CClF₂CCl₂CCl₃, and the conversion of CF₃CH₂CF₃ to a mixture of CCl₂═CHCF₃, and CCl₂═CClCF₃.

Of note is a catalytic process for producing a mixture containing 1,1-dichloro-3,3,3-trifluoro-1-propene (i.e., CCl₂═CHCF₃ or HCFC-1223za) and 1,1,2-trichloro-3,3,3-trifluoro-1-propene (i.e., CCl₂═CClCF₃ or CFC-1213xa) by the chlorodefluorination of 1,1,1,3,3,3-hexafluoropropane (i.e., CF₃CH₂CF₃ or HFC-236fa) by reaction of HFC-236fa with HCl in the vapor phase in the presence of the catalysts of this invention. The reaction is preferably conducted from about 275° C. to about 450° C., more preferably about 300° C. to about 400° C. with a molar ratio of HCl to HFC-236fa of preferably from about 3:1 to about 20:1. Of note are contacts times of from about 1 second to about 40 seconds. Oxygen in the form of air or co-fed with an inert diluent such as nitrogen, helium, or argon may be added along with the reactants or as a separate catalyst treatment, if desired.

The reaction products obtained by the processes of this invention can be separated by conventional techniques, such as with combinations including, but not limited to, scrubbing, decantation, or distillation. Some of the products of the various embodiments of this invention may form one or more azeotropes with each other or with HF.

The processes of this invention can be carried out readily using well known chemical engineering practices.

### Utility

Several of the reaction products obtained through use of the catalysts disclosed herein will have desired properties for direct commercial use. For example, CH₂F₂ (HFC-32), CHF₂CF₃ (HFC-125), CHF₂CH₃ (HFC-152a), CH₂FCF₃ (HFC-134a), CF₃CH₂CF₃ (HFC-236fa), and CF₃CH₂CHF₂ (HFC-245fa) find application as refrigerants, CH₂FCF₃ (HFC-134a) and CF₃CHFCF₃ (HFC-227ea) find application as propellants, CH₃CHF₂ (HFC-152a) and CF₃CH₂CHF₂ (HFC-245fa) find application as blowing agents, and CHF₂CF₃ (HFC-125), CF₃CH₂CF₃ (HFC-236fa), and CF₃CHFCF₃ (HFC-227ea) find application as fire extinguishants.

Other reaction products obtained through the use of this invention are used as chemical intermediates to make useful products. For example, CCl₃CF₃ (CFC-113a) can be used to prepare CFC-114a which can then be converted to CH₂FCF₃ (HFC-134a) by hydrodechlorination. Similarly, CF₃CCl₂CF₃ (CFC-216aa) can be used to prepare CF₃CH₂CF₃ (HFC-236fa) by hydrodechlorination and CF₃CCl═CF₂ (CFC-1215zc) can be used to prepare CF₃CH₂CHF₂ (HFC-245fa) by hydrogenation.

Without further elaboration, it is believed that one skilled in the art can, using the description herein, utilize the present invention to its fullest extent. The following specific embodiments are, therefore, to be construed as merely illustrative, and do not constrain the remainder of the disclosure in any way whatsoever.

### EXAMPLES

### Catalyst Characterization

Energy Dispersive Spectroscopy (EDS) and Transmission Electron Microscopy ITEM)

In these studies, the crystallites were analyzed using a Philips CM-20 high-resolution transmission electron microscope operated at an accelerating voltage of 200 kV and configured with an Oxford windowless EDS system with a Si(Li) elemental detector. In the EDS analyses, electron-transparent thin sections of samples were used to minimize sample thickness effects such as fluorescence. Also, due to the similarity of their atomic masses, the X-ray absorption cross-sections for Cr and Cu were assumed to be the same (see the discussion by Zaluzec on pages 121 to 167 in Introduction to Analytical Electron Microscopy edited by J. J. Hren, J. I. Goldstein, and D. C. Joy (Plenum Press, New York, 1979). The samples were dispersed on Al grids to ensure that the Cu detected by the EDS analysis truly represented the Cu contained in the samples.

X-Ray Absorption Spectroscopy (XAS) and X-Ray Powder Diffraction (XRD)

XRD data were obtained and analyzed according to methods described by Warren in X-Ray Diffraction (Addison-Wesley, Reading, Mass., 1969). XAS data were obtained at beamline 5BMD, DND-CAT, of the Advanced Photon Source, Argonne National Laboratory. XAS data were obtained and analyzed using the methods described in Koningsberger and Prins in X-ray Absorption: Principles, Applications, Techniques of EXAFS, SEXAFS and XANES (John Wiley & Sons, New York, 1988). Spectra were obtained for the K edges of Cr, and Cu. Cr edges were obtained in transmission geometry, while Cu edges were obtained in fluorescence mode, due to their low concentrations.

Oxidation states were obtained by fitting of sample near edge spectra to those of standards with known oxidation states.

Use of the Advanced Photon Source for acquiring XAS data was supported by the U.S. Department of Energy, Office of Basic Energy Sciences, under Contract No. W-31-109-Eng-38.

### Catalyst Preparations

### Comparative Preparation Example 1

### Preparation of 100% Chromium Catalyst

A solution of 400 g Cr(NO₃)₃[9(H₂O)] (1.0 mole) in 1000 mL of deionized water was treated dropwise with 477 mL of 7.4M aqueous ammonia raising the pH to about 8.5. The slurry was stirred at room temperature overnight. After re-adjusting the pH to 8.5 with ammonia, the mixture was poured into evaporating dishes and dried in air at 120° C. The dried solid was then calcined in air at 400° C.; the resulting solid weighed 61.15 g. The catalyst was pelletized (−12 to +20 mesh, 1.68 to 0.84 mm)) and 28.2 g (20 mL) was used in Comparative Example 1.

### Preparation Example 1

### Preparation of 99% Chromium/1% Copper Catalyst

To a one liter beaker containing 261.0 g Cr(NO₃)₃[9(H₂O)] (0.652 mole) and 1.46 g Cu(NO3)2[2.5H2O] 0.0063 mole) was added 100 mL of deionized water. The slurry was placed on a stirring hot plate in a fume-hood and heated while stirring until oxides of nitrogen started to evolve. The beaker containing the paste-like material was placed in a furnace in the fume-hood after removing the stirrer. The temperature of the furnace was raised to 150° C. at the rate of 10 degrees/min and then to 550° C. at the rate of 1 degree/minute. It was held at 550° C. for an additional 10 hours. The resulting solid was pelletized (−12 to +20 mesh, 1.68 to 0.84 mm)) and 12.6 g (8.0 mL) was used in Examples 1 and 8.

### Preparation Example 2

### Preparation of 99% Chromium/1% Copper Catalyst

In a 2000 mL beaker was placed 400.2 g Cr(NO₃)₃[9(H₂O)] (1.0 mole) and 1.64 g CuCl2 (0.012 mole). To the solids was added 1000 mL deionized water. The mixture was stirred and when the dissolution was complete, the pH of the solution was raised from 2.0 to 8.0 by drop-wise addition of 8 molar aqueous ammonium hydroxide. The precipitated slurry was stirred for 24 hours at room temperature. It was then dried at 120-130° C. overnight and calcined at 450° C. for an additional 24 hours in air. The resulting solid was pelletized (−12 to +20 mesh, 1.68 to 0.84 mm)) and 11.0 g (8.0 mL) was used in Examples 2 and 9.

### Preparation Example 3

### Preparation of 99% Chromium/1% Copper Catalyst

In a 3000 mL beaker was placed 500.0 g Cr(NO₃)₃[9(H₂O)] (1.25 moles) and 3.05 g Cu(NO3)2[2.5H2O (0.013 mole). To the solids was added 1200 mL deionized water. The mixture was stirred and when the dissolution was complete, the pH of the solution was raised from 2.4 to 8.5 by drop-wise addition of 300 mL of 8 molar aqueous ammonium hydroxide. The precipitated slurry was stirred for 24 hours at room temperature. It was then dried at 110-120° C. overnight and calcined at 500° C. for an additional 24 hours in air. The resulting solid was pelletized (−12 to +20 mesh, 1.68 to 0.84 mm)) and 16.0 g (8.0 mL) was used in Examples 3 and 10.

### Preparation Example 4

### Preparation of 98% Chromium/2% Copper Catalyst

Preparation Example 1 was substantially repeated except that the amount of chromium(III) nitrate was 258.0 g (0.645 mole) and the amount of copper (II) nitrate was 2.9 g (0.0125 mole). The resulting solid was pelletized (−12 to +20 mesh, 1.68 to 0.84 mm)) and 12.6 g (8.0 mL) was used in Examples 4 and 11.

### Preparation Example 5

### Preparation of 98% Chromium/2% Copper Catalyst

Preparation Example 2 was substantially repeated with 400.2 g chromium (III) nitrate (1.0 mole) and 3.31 g (0.0246 mole) copper (II) chloride. The solid, calcined in air at 450° C. for 24 hours, was pelletized (−12 to +20 mesh, 1.68 to 0.84 mm)) and 10.9 g (8.0 mL) was used in Examples 5 and 12.

### Preparation Example 6

### Preparation of 98% Chromium/2% Copper Catalyst

In a 3000 mL beaker was placed 500.0 g Cr(NO₃)₃[9(H₂O)] (1.1.25 mole) and 6.1 g Cu(NO3)2[2.5H2O (0.0262 mole). To the solids was added 1200 mL deionized water. The mixture was stirred and when the dissolution was complete, the pH of the solution was raised from 2.4 to 8.2 by drop-wise addition of 300 mL 8 molar aqueous ammonium hydroxide. The precipitated slurry was stirred for 24 hours at room temperature. It was then dried at 110-120° C. overnight and calcined at 500° C. for an additional 24 hours in air. The resulting solid was pelletized (−12 to +20 mesh, 1.68 to 0.84 mm)) and 14.9 g (8.0 mL) was used in Examples 6 and 13 as the catalyst.

### Preparation Example 7

### Preparation of 95% Chromium/5% Copper Catalyst

Preparation Example 1 was substantially repeated except that the amount of chromium (III) nitrate was 250.0 g (0.625 mole) and the amount of copper (II) nitrate was 7.3 g (0.314 mole). The resulting solid was pelletized (−12 to +20 mesh, 1.68 to 0.84 mm)) and 11.9 g (8.0 mL) was used in Examples 7 and 14.

### Preparation Examples 8-9

### Preparation of 95% Chromium/5% Copper Catalyst

Preparation Example 6 was substantially repeated except that the amounts of chromium (III) nitrate and copper (II) were adjusted to produce a catalyst having a ratio of chromium to copper of 95/5. The solid dried at 110-120° C. overnight was divided into two portions. One portion was calcined at 500° C. and another portion was calcined at 900° C. A 35.8 g (25.0 ml) portion, calcined at 500° C. and pelletized to −12 to +20 mesh (1.68 to 0.84 mm), was used in Examples 8 and 15. Similarly a 48.1 g (25.0 ml) portion, calcined at 900° C. and pelletized to ±12 to +20 mesh (1.68 to 0.84 mm), was used in Examples 9 and 16.

### Examples 1-7 and Comparative Example 1

### General Procedure for Fluorination and Chlorofluorination

A weighed quantity of pelletized catalyst was placed in a ⅝ inch (1.58 cm) diameter Inconel™ nickel alloy reactor tube heated in a fluidized sand bath: The tube was heated from 50° C. to 175° C. in a flow of nitrogen (50 cc/min; 8.3(10)⁻⁷ m³/sec) over the course of about one hour. HF was then admitted to the reactor at a flow rate of 50 cc/min (8.3(10)⁻⁷ m³/sec). After 0.5 to 2 hours the nitrogen flow was decreased to 20 cc/min (3.3(10)⁻⁷ m³/sec) and the HF flow increased to 80 cc/min (1.3(10)⁻⁶ m³/sec); this flow was maintained for about 1 hour. The reactor temperature was then gradually increased to 400° C. over 3 to 5 hours. At the end of this period, the HF flow was stopped and the reactor cooled to 300° C. under 20 sccm (3.3(10)⁻⁷ m³/sec) nitrogen flow. CFC-1213xa was fed from a pump to a vaporizer maintained at about 118° C. For fluorinations, the CFC-1213xa vapor was combined with the appropriate molar ratios of HF in a 0.5 inch (1.27 cm) diameter Monel™ nickel alloy tube packed with Monel™ turnings. The mixture of reactants then entered the reactor. The HF/1213xa molar ratio was 20 and the contact time was 5 seconds for Examples 1-7. For chlorofluorinations, the CFC-1213xa vapor was combined with the appropriate molar ratios of HF and chlorine. The HF/1213xa/chlorine molar ratio was 20/1/4 for all runs and the contact time was 5 seconds for Examples 8-14 and 30 seconds for Examples 15-16. The reactions were conducted at a nominal pressure of one atmosphere. Analytical data for identified compounds is given in units of GC area %. Small quantities of other unidentified products were present.

### General Procedure for Fluorocarbon Product Analysis

The following general procedure is illustrative of the method used for analyzing the products of fluorination and chlorofluorination reactions. Part of the total reactor effluent was sampled on-line for organic product analysis using a gas chromatograph equipped a mass selective detector (GC-MS). The gas chromatography was accomplished with a 20 ft. (6.1 m) long×⅛ in. (0.32 cm) diameter tubing containing Krytox® perfluorinated polyether on an inert carbon support. The helium flow was 30 mL/min (5.0(10)⁻⁷ m³/sec). Gas chromatographic conditions were 60° C. for an initial hold period of three minutes followed by temperature programming to 200° C. at a rate of 6° C./minute.

The bulk of the reactor effluent containing organic products and also inorganic acids such as HCl and HF was treated with aqueous caustic prior to disposal.

Legend

| | |
|---|---|
| 214ab is CF₃CCl₂CCl₂F | 215aa is CF₃CCl₂CClF₂ |
| 215bb is CCl₂FCClFCF₃ | 216aa is CF₃CCl₂CF₃ |
| 216ca is CClF₂CF₂CClF₂ | 216cb is CF₃CF₂CCl₂F |
| 216ba is CClF₂CClFCF₃ | 217ba is CF₃CClFCF₃ |
| 217ca is CF₃CF₂CClF₂ | 225da is CF₃CHClCClF₂ |
| 226da is CF₃CHClCF₃ | 1213xa is CF₃CCl═CCl₂ |
| 1214 is C₃Cl₂F₄ | 1215xc is CF₃CCl═CF₂ |

### Examples 1-7

### Fluorination of 1213xa

The fluorination of CFC-1213xa was carried out at various temperatures using catalysts prepared according to Catalyst Preparation Examples 1-7. The analytical results are shown in Table 1.

TABLE 1

| Ex.. | Cat | | | | | | | | | |
|---|---|---|---|---|---|---|---|---|---|---|
| No. | Prep. | T ° C. | 1215xc | 226da | 216aa | 1214 | 225da | 215aa | 215bb | 1213xa |
| 1 | 1 | 280 | 17.9 | 64.8 | 5.0 | 4.0 | 3.5 | 1.2 | ND | 2.8 |
| | | 320 | 8.3 | 85.0 | 3.1 | 1.8 | 0.6 | 0.2 | ND | 0.8 |
| 2 | 2 | 280 | 3.1 | 90.9 | 3.2 | 0.5 | 0.9 | 0.6 | ND | 0.4 |
| | | 300 | 1.3 | 93.5 | 3.8 | 0.2 | 0.2 | 0.4 | ND | 0.2 |
| | | 320 | 1.7 | 93.7 | 3.3 | 0.3 | 0.2 | 0.2 | ND | 0.2 |
| 3 | 3 | 280 | 25.0 | 57.1 | 4.5 | 4.9 | 5.2 | 1.1 | ND | 2.1 |
| | | 320 | 8.5 | 83.3 | 4.4 | 2.1 | 0.6 | 0.3 | ND | 0.8 |
| 4 | 4 | 280 | 53.3 | 7.3 | 2.7 | 11.5 | 3.6 | 2.5 | 1.2 | 17.7 |
| | | 320 | 62.2 | 12.3 | 2.5 | 13.3 | 2.7 | 0.9 | ND | 6.0 |
| 5 | 5 | 280 | 53.7 | 12.8 | 2.4 | 12.0 | 5.5 | 1.9 | ND | 11.1 |
| | | 320 | 59.4 | 14.2 | 1.7 | 13.9 | 3.8 | 0.1 | ND | 6.3 |
| | | 350 | 56.7 | 21.7 | 3.5 | 11.0 | 1.8 | ND | ND | 3.4 |
| 6 | 6 | 280 | 51.8 | 23.6 | 3.9 | 7.8 | 3.9 | 1.5 | ND | 7.2 |
| | | 320 | 49.0 | 29.0 | 4.0 | 10.3 | 2.4 | 0.4 | ND | 4.8 |
| 7 | 7 | 280 | 28.9 | 0.6 | 1.0 | 16.0 | 0.2 | 1.5 | 1.5 | 50.3 |
| | | 320 | 51.3 | 0.8 | 2.2 | 19.6 | 0.6 | 2.4 | 0.3 | 22.7 |
| | | 350 | 68.5 | 0.9 | 2.8 | 16.3 | ND | 0.7 | ND | 9.6 |

| | | | | | | | | | |
|---|---|---|---|---|---|---|---|---|---|
| Comp. Ex. 1 | 300 | ND | 89.7 | 7.8 | ND | ND | ND | ND | ND |

ND = not detected

Examination of the data in the fluorination examples above show that the fluorine content of the starting CFC-1213xa is increased to produce CFC-1215xc, HCFC-226da as well as other useful products containing a higher fluorine content than the starting material by using the catalysts of this invention.

### Examples 8-16

### Chlorofluorination of 1213xa

The chlorofluorination of CFC-1213xa was carried out at various temperatures using catalysts prepared according to Catalyst Preparation Examples 1-9. The analytical results are shown in Table 2.

TABLE 2

| Ex.. | Cat | | | | | | | | | | | | |
|---|---|---|---|---|---|---|---|---|---|---|---|---|---|
| No. | Prep. | T ° C. | 217ba | 217ca | 1215xc | 226da | 216aa | 216ba | 216cb | 215aa | 215bb | 214ab | 1214 |
| 8 | 1 | 280 | 0.7 | ND | 0.9 | 2.4 | 14.4 | 4.8 | 0.6 | 63.4 | 8.5 | 3.2 | 0.3 |
| | | 320 | 3.4 | 0.3 | 1.0 | 2.4 | 36.3 | 14.8 | 1.2 | 38.9 | 1.4 | ND | ND |
| | | 375 | 5.8 | 1.3 | 0.3 | 1.4 | 60.2 | 13.7 | 0.4 | 16.7 | ND | ND | ND |
| 9 | 2 | 280 | 0.4 | ND | 0.4 | 1.4 | 13.2 | 7.6 | 0.8 | 61.0 | 13.8 | ND | ND |
| | | 320 | 1.4 | 0.4 | 0.2 | 1.4 | 31.1 | 23.3 | 1.0 | 41.1 | 0.1 | ND | ND |
| | | 375 | 3.2 | 1.2 | 0.1 | 0.8 | 59.3 | 16.7 | 0.2 | 18.4 | 0.1 | ND | ND |
| 10 | 3 | 320 | 2.4 | 0.4 | 0.3 | 0.8 | 32.8 | 26.6 | 2.0 | 33.5 | 1.1 | ND | ND |
| | | 350 | 2.9 | 1.1 | 0.3 | 0.5 | 42.3 | 26.5 | 1.4 | 24.8 | ND | ND | ND |
| | | 375 | 3.4 | 1.6 | 0.1 | 0.5 | 53.6 | 21.8 | 0.5 | 18.5 | ND | ND | ND |
| 11 | 4 | 280 | 0.2 | ND | 1.7 | 0.4 | 11.0 | 2.3 | 1.4 | 26.5 | 33.6 | 18.2 | 4.7 |
| | | 320 | 0.4 | ND | 0.9 | 0.5 | 21.0 | 12.1 | 1.9 | 41.8 | 20.4 | 0.8 | 0.1 |
| | | 350 | 0.5 | 0.2 | 0.6 | 0.4 | 28.1 | 21.2 | 2.5 | 36.8 | 9.4 | 0.1 | ND |
| 12 | 5 | 350 | 0.2 | 0.2 | 0.2 | 0.2 | 18.4 | 28.8 | 1.7 | 45.5 | 4.7 | ND | ND |
| | | 375 | 0.3 | 0.5 | 0.2 | 0.1 | 24.4 | 30.6 | 1.6 | 41.4 | 0.7 | ND | ND |
| | | 400 | 0.6 | 0.9 | 0.2 | 0.1 | 31.5 | 28.5 | 1.2 | 36.7 | 0.2 | ND | ND |
| 13 | 6 | 320 | 0.3 | 0.2 | 0.2 | 0.2 | 16.3 | 27.7 | 2.4 | 41.7 | 10.3 | ND | ND |
| | | 350 | 0.9 | 0.8 | 0.3 | 0.2 | 26.7 | 33.1 | 2.0 | 33.9 | 2.0 | ND | ND |
| | | 375 | 2.2 | 1.8 | 0.1 | 0.1 | 44.3 | 28.4 | 0.8 | 21.8 | 0.4 | ND | ND |
| 14 | 7 | 320 | ND | ND | 1.1 | 0.1 | 8.5 | 4.3 | 1.5 | 39.6 | 36.0 | 7.8 | 1.0 |
| | | 350 | 0.1 | 0.1 | 0.9 | 0.1 | 10.9 | 10.4 | 2.0 | 42.9 | 30.9 | 1.6 | 0.3 |
| | | 400 | 0.1 | 0.1 | 0.6 | ND | 12.4 | 19.8 | 1.9 | 46.8 | 17.9 | 0.3 | 0.1 |
| 15 | 8 | 280 | ND | ND | 0.8 | ND | 3.5 | 0.9 | 0.5 | 26.7 | 36.0 | 26.5 | 4.6 |
| | | 320 | ND | ND | 1.9 | ND | 6.7 | 11.8 | 0.8 | 49.8 | 27.2 | 0.7 | 0.3 |
| | | 425 | ND | ND | 0.9 | 0.2 | 5.5 | 25.7 | 0.7 | 59.1 | 5.9 | 0.1 | 0.2 |
| 16 | 9 | 280 | ND | ND | 0.3 | ND | 2.9 | 0.4 | 0.6 | 20.2 | 47.3 | 25.9 | 1.9 |
| | | 320 | ND | ND | 0.3 | ND | 3.8 | 1.4 | 1.0 | 29.3 | 48.4 | 14.3 | 1.1 |
| | | 425 | ND | ND | 0.3 | ND | 5.1 | 12.8 | 1.4 | 50.8 | 28.1 | 0.6 | 0.2 |

### Table 2

### Continued

Examination of the data in the chlorofluorination examples above show that the fluorine content of the starting CFC-1213xa is increased to produce CFC-216aa and CFC-216ba as well as other useful products containing a higher fluorine content than the starting material by using the catalysts of this invention.

The examples above illustrate use of the catalysts of this invention to increase the fluorine content of a compound. Using the catalysts of this invention, the fluorine distribution in a halogenated hydrocarbon ° compound may be changed by isomerization or disproportionation or the fluorine content of a compound may be decreased by dehydrofluorination or by reaction with hydrogen chloride in a manner analogous to the teachings of International Publication No. WO 2004/018093 A2, which is incorporated herein by reference.

What is claimed is:

1. A crystalline alpha-chromium oxide where from about 0.05 atom % to about 5 atom % of the chromium atoms in the alpha-chromium oxide lattice are replaced by divalent copper atoms.

2. A chromium-containing catalyst composition comprising as a chromium-containing component the crystalline copper-substituted alpha-chromium oxide of claim 1.

3. A chromium-containing catalyst composition comprising a chromium-containing component prepared by treating the crystalline copper-substituted alpha-chromium oxide of claim 1 with a fluorinating agent.

4. A process for changing the fluorine distribution in a hydrocarbon or a halogenated hydrocarbon in the presence of a catalyst, characterized by:
using as the catalyst a composition comprising at least one chromium-containing component selected from the group consisting of the crystalline copper-substituted alpha-chromium oxide of claim 1 and a crystalline copper-substituted alpha-chromium oxide of claim 1 which has been treated with a fluorinating agent.

5. The process of claim 4 wherein the fluorine content of a halogenated hydrocarbon compound or an unsaturated hydrocarbon compound is increased by reacting said compound with hydrogen fluoride in the vapor phase in the presence of said catalyst composition.

6. The process of claim 4 wherein the fluorine content of a halogenated hydrocarbon compound or a hydrocarbon compound is increased by reacting said compound with HF and Cl₂ in the vapor phase in the presence of said catalyst composition.

7. The process of claim 4 wherein the fluorine distribution in a halogenated hydrocarbon compound is changed by isomerizing said halogenated hydrocarbon compound in the presence of said catalyst composition.

8. The process of claim 4 wherein the fluorine distribution in a halogenated hydrocarbon compound is changed by disproportionating said halogenated hydrocarbon compound in the vapor phase in the presence of said catalyst composition.

9. The process of claim 4 wherein the fluorine content of a halogenated hydrocarbon compound is decreased by dehydrofluorinating said halogenated hydrocarbon compound in the presence of said catalyst composition.

10. The process of claim 4 wherein the fluorine content of a halogenated hydrocarbon compound is decreased by reacting said halogenated hydrocarbon compound with hydrogen chloride in the vapor phase in the presence of said catalyst composition.

11. A method for preparing a composition comprising the crystalline copper-substituted alpha-chromium oxide of claim 1, comprising:
(a) co-precipitating a solid by adding ammonium hydroxide to an aqueous solution of a soluble copper salt and a soluble trivalent chromium salt that contains at least three moles of nitrate per mole of chromium in the solution and has a copper concentration of from about 0.05 atom % to about 5 atom % of the total concentration of copper and chromium in the solution; and after at least three moles of ammonium per mole of chromium in the solution has been added to the solution;
(b) collecting co-precipitated solid formed in (a);
(c) drying the collected solid; and
(d) calcining the dried solid.

12. The method of claim 11 wherein the soluble copper salt is a divalent copper salt.

13. The method of claim 12 wherein the soluble copper and chromium salts are nitrates or hydrated nitrates

14. The method of claim 12 wherein more than three moles of ammonium nitrate per mole of chromium is present in the aqueous solution.

15. A method for preparing a composition comprising the crystalline copper-substituted alpha-chromium oxide of claim 1, comprising:
(a) preparing an aqueous solution of a soluble copper salt and a soluble trivalent chromium salt that contains a copper concentration of from about 0.05 atom % to about 5 atom % of the total concentration of copper and chromium in the solution;
(b) evaporating the solution to dryness; and
(c) calcining the dried solid.
